(19) United States
(12) Patent Application Publication
(10) Pub. No.: US 2015/0321164 A1
(43) Pub. Date: Nov. 12, 2015

(54) METHOD FOR SYNTHESIZING AND SCREENING LEAD COMPOUND AND REAGENT TESTING KIT

(71) Applicant: HITGEN LTD., Chengdu, Sichuan (CN)

(72) Inventors: Jin LI, Chengdu, Sichuan (CN); Yang JIANG, Chengdu, Sichuan (CN); Shi CHEN, Chengdu, Sichuan (CN); Qingxi QU, Chengdu, Sichuan (CN); Jinqiao WAN, Chengdu, Sichuan (CN); Lina ZHONG, Chengdu, Sichuan (CN); Xiaohu GE, Chengdu, Sichuan (CN); Qi HUANG, Chengdu, Sichuan (CN)

(21) Appl. No.: 14/653,729

(22) PCT Filed: Dec. 18, 2013
(86) PCT No.: PCT/CN2013/089873

§ 371 Date: Jun. 18, 2015

(30) Foreign Application Priority Data
Dec. 20, 2012 (CN) 201210555548.3

(51) Int. Cl. B01J 19/00; C12Q 1/68

(52) U.S. Cl. CPC B01J 19/0046; C12Q 1/6874; B01J 2219/00722

(57) ABSTRACT

A method for synthesizing and screening a lead compound, comprising the following steps: (1) retrieving raw materials: retrieving an i-number of synthetic blocks and an (i+2)-number of single-stranded DNA fragments; (2) synthesizing a compound by using a combinatorial chemistry method, acquiring a library of a single-stranded DNA-marked compound; (3) screening: screening the library of the DNA-marked compound; and, (4) sequencing: retrieving the DNA-marked compound screened in step (3), and sequencing the DNA on the DNA-marked compound, where the synthesis blocks and reaction mechanism of the compound can be determined on the basis of the DNA sequencing. Also disclosed are a synthesis and screening reagent testing kit for the lead compound and a combinatorial chemistry library.

### FIELD OF THE INVENTION

The present invention relates to the field of chemical synthesis, in particular to a method for synthesizing and screening lead compounds in drug discovery research and a kit.

### BACKGROUND OF THE INVENTION

Since the late 1980s, with the breakthrough of molecular biological studies and the development of high throughout technologies, more and more new molecular entities are required for the development of new drugs, and scientists have turned their attention from seeking natural products to synthesizing a large number of compound groups or groups of compounds, i.e., chemical libraries. The chemical libraries are composed of many organic compounds of different attributes. The combinatorial chemistry method is a technology for synthesizing chemical libraries. By this technology, different series of synthetic building blocks, i.e., reagents, are arranged orderly to form a large series of diversified molecular entity groups. The combinatorial chemistry method is often referred to as a number's game, i.e., a method on how to arrange numerous synthetic building blocks combinatorially to form large of reaction products, chemical compounds. Theoretically, the total number N of reaction products from the combinatorial synthesis is determined by two factors, i.e., the number b of synthetic building blocks in each step and the number x of synthesis steps. For example, for a linear combination reaction having three steps, if the number of reactants in each step is b1, b2 and b3, respectively, the theoretical total number of reaction products is N=b1*b2*b*3. An objective of the combinatorial chemistry studies is how to effectively obtain all resulting products N of this reaction scheme. Recently, from the solid-phase synthesis to the rapid liquid-phase parallel synthesis, the combinatorial chemistry method has achieved a breakthrough in terms of synthesis methods. Several common synthesis methods include solid-phase organic synthesis and liquid-phase organic synthesis. The solid-phase organic synthesis includes mixed splitting and parallel synthesis, while the liquid-phase organic synthesis includes multi-component liquid-phase synthesis and functional group conversion.

In a chemical library established by the combinatorial chemistry synthesis technology, there are thousands of or even billions of resulting products. It is impossible to purify, separate and identify the resulting products one by one as for the typical organic synthesis. The High Throughput Screening (HTS) technology refers to a technology system where, based on the experimental methods in the molecular level and the cell level, using microplates as experimental tool supports, executing an experiment process by an automatic operating system, collecting the data of experimental results by a sensitive and rapid detecting instrument, and analyzing and processing the experimental data by a computer, thousands of and millions of samples are detected rapidly and the operation of the whole system is supported by a corresponding database. The high throughput screening method greatly improves the speed and efficiency of screening small-molecule compounds, and may screen, from a combinatorial chemistry library, compounds acting on target molecules. However, after screening compounds from a chemical library by a conventional high throughput screening method, it is very difficult to purify target compounds and determine the structure thereof, and it is time-consuming and costly. With the expansion of the compound library, this becomes more difficult.

To solve this problem, Patent Application No. 95193518.6, entitled “Complex Combinatorial Chemical Libraries Encoded with Tags”, disclosed a method, where at each stage of the synthesis, a support (for example, a particle) upon which a compound is being synthesized, is uniquely tagged to define a particular event, usually chemical reagents, associated with the synthesis of the compound on the support. The tagging is accomplished using identifier molecules which record the sequential events to which the supporting particle is exposed during synthesis, thus providing a reaction history for the compound produced on the support. However, no technical solution for realizing the method is provided in this application.

In the prior art, it is reported that oligonucleotides are used to tag synthesis units of compounds. As a double-stranded DNA is more stable than a single-stranded DNA under normal conditions according to the common knowledge in the biological field, double-stranded oligonucleotides are usually selected to tag the synthesis units of compounds.

As another example, Patent EP0643778, entitled “encoded combinatorial chemical libraries”, disclosed a method of using single-stranded oligonucleotides to tag amino acids or polypeptides; U.S. Pat. No. 7,935,658, entitled “methods for synthesis of encoded libraries”, disclosed a method of using single-stranded DNA fragments to tag synthetic building blocks to form compound libraries; and Patent WO/2010/094036, entitled “METHODS OF CREATING AND SCREENING DNA-ENCODED LIBRARIES”, disclosed a method of using oligonucleotides to tag compounds to form compound libraries, where the oligonucleotides were double-stranded DNA with hairpin structure.

However, when the double-stranded DNA is used to tag synthetic building blocks or compounds, during linking and extension, the double-stranded DNA is likely to be cross-linked to form a curly tertiary structure. Therefore, during sequencing, it is required to perform unlinking, and the operation is relatively complicated. When the double-stranded DNA is used to tag a linear combination reaction having more than three steps, the result of sequencing of the double-stranded DNA has a large error. Therefore, it is necessary to find a new tagging method with simple operation and more accurate results.

### SUMMARY OF THE INVENTION

To solve the above problems, the present invention provides a kit and method for synthesizing and screening lead compounds, and a new combinatorial chemistry library.

### Definition

Synthetic building blocks: also called synthons, refer to small-molecule compounds which have various physicochemical properties and specific biochemical properties and must be used in the development of new drugs (western medicines, pesticides).

Lead compounds: refer to compounds, obtained by various approaches and means, which have a certain bioactivity and a chemical structure and are used for further structural reconstructions and modifications, being the starting point of the development of modern new drugs.

Reaction mechanism: the process of a chemical reaction.

Linking in series: a number of fragments of single-stranded DNA sequences are successively linked endpoint by endpoint, without any branch at the linkage.

The present invention provides a method for synthesizing and screening lead compounds, including the following steps of:

(1) Preparing raw materials, i.e., i synthetic building blocks and (i+2) single-stranded DNA fragments, where, the (i+2) single-stranded DNA fragments comprise i tag sequences, a start sequence and a terminal sequence, and the i tag sequences specifically tag the i synthetic building blocks, respectively, where i=1, 2, 3 . . . n;

(2) Synthesizing a compound library by combinatorial chemistry method:

a, preparing initial synthetic building blocks: selecting 1 to i synthetic building blocks, linking in series one end of the start sequence to a synthetic building block and the other end of the start sequence to a specific tag sequence of the synthetic building block to obtain 1 to i initial synthetic building blocks tagged with single-stranded DNA with a free 3′-end;

b, synthesizing compounds by reacting the initial synthetic building blocks obtained in step a and the 1 to i synthetic building blocks in a manner of linear combination, wherein, during synthesis, once a new synthetic building block is added, a specific tag sequence of this new synthetic building block is linked in series to the free end of the single-stranded DNA linked to the initial synthetic building blocks such that the single-stranded DNA is gradually lengthened; at the end of synthesis, the terminal sequence is linked in series to the free end of the single-stranded DNA to obtain a single-stranded DNA-encoded compound library;

(3) Screening: screening the DNA-encoded compound library to select target compounds; and

(4) Sequencing: sequencing the DNA of the target compounds screened in step (3), and determining synthetic building blocks and reaction mechanisms for the target compounds.

The start sequence in step (1) includes poly-adenosine. Preferably, the poly-adenosine includes 12 to 20 adenosines.

The length of the tag sequences in step (1) is not less than 6 bp. Preferably, the length of the tag sequences is 9 bp.

In step (2), during synthesis, the pH is 8-12 and the temperature is 0-30° C.

In step (1), a ribonucleotide is linked to the 3′-end of the tag sequences in step (1), and the ribonucleotide is cytidine.

In step (2), a method for linking the start sequence to the initial synthetic building blocks in step a is as follows:

performing amination to the start sequence, performing carboxylation, sulfhydrylization or alkynylation to the initial synthetic building blocks, and reacting the start sequence with the initial synthetic building blocks.

In step (2), a method for linking the start sequence to the tag sequences, linking the tag sequences or linking the tag sequences to the terminal sequence is as follows: phosphorylating the 5′-end of the single-stranded DNA with polynucleotide kinase and then linking using RNA ligase. The polynucleotide kinase is T4 polynucleotide kinase, and the RNA ligase is T4 RNA ligase.

The screening method in step (3) is one based on a receptor-ligand specific reaction.

The present invention provides a kit for synthesizing and screening lead compounds, including the following components:

1) i synthetic building blocks and (i+2) single-stranded DNA fragments, where the (i+2) single-stranded DNA fragments comprise i tag sequences, a start sequence and a terminal sequence, and the i tag sequences specifically tag the i synthetic building blocks, respectively, where i=1, 2, 3 . . . n;

2) a reagent for linking the start sequence-initial synthetic building blocks, a reagent for combinatorial chemistry method and a reagent for linking the single-stranded DNA fragments;

3) a reagent for screening compounds; and

4) a reagent for DNA sequencing.

The start sequence in component 1) includes poly-adenosine. Preferably, the poly-adenosine includes 12 to 20 adenosines.

The length of the tag sequences in component 1) is not less than 6 bp. Preferably, the length of the tag sequences is 9 bp.

A ribonucleotide is linked to the 3′-end of the tag sequences in component 1), and the ribonucleotide is cytidine.

The reagent for linking the start sequence in component 2) to the synthetic building blocks comprises a reagent for amination of the start sequence, and a reagent for carboxylation, sulfhydrylization or alkynylation of the synthetic building blocks.

The reagent for linking single-stranded DNA fragments in component 2) includes polynucleotide kinase and RNA ligase.

Preferably, the polynucleotide kinase is T4 polynucleotide kinase, and the RNA ligase is T4 RNA ligase.

The present invention provides a combinatorial chemistry library, which is a combinatorial chemistry library synthesized by combinatorial chemistry method using synthetic building blocks as raw materials, wherein a fragment of a single-stranded DNA sequence is tagged for each compound; and the single-stranded DNA sequence has a following structure: a start sequence-i tag sequences-a terminal sequence, the i tag sequences specifically tag i synthetic building blocks used during the combinatorial chemistry synthesis, and the order of the i tag sequences is the same as an order of adding the synthetic building blocks during the combinatorial chemistry synthesis.

The length of the tag sequences is not less than 6 bp. Preferably, the length of the tag sequences is 9 bp.

When the length of the tag sequences is 6 bp, 4096 single-stranded DNA fragments of different sequences may be obtained, and there are thousands of synthetic building blocks encoded with the DNA fragments and used for preparing combinatorial chemistry libraries, so that the requirements on the synthesis and screening of the majority of compounds can be met. When the length of the tag sequences is 9 bp, 262144 single-stranded DNA fragments of different sequences may be obtained, and there are millions of, up to 262144, synthetic building blocks encoded with these DNA fragments and used for preparing combinatorial chemistry libraries, so that the requirements on the compound synthesis and screening may be completely met. If the length of the tag sequences is longer, more synthetic building blocks may be encoded, and the prepared combinatorial chemistry libraries are larger. However, correspondingly, the cost is higher. Comprehensively considering the capacity of a library and the cost, the length of the tag sequences is most preferably 9 bp.

In the context of the present invention, using single-stranded DNA to tag synthetic building blocks, the single-stranded DNA will not be complementary to form double strands during linking, and thus is stable in structure and difficult to be cross-linked. Thus, it is not required to perform unlinking during sequencing, the operation is simple and rapid, and the results are more accurate. Therefore, the method provided by the present invention may include multiple linear combination reaction steps, the synthesized compound library has high diversity and large capacity, and it is easy to obtain target compounds by synthesis and determine their synthetic building blocks, reaction mechanisms and chemical structures, so that a large number of target compounds may be synthesized rapidly. In conclusion, the method provided by the present invention is a method for synthesizing and screening lead compound libraries, with high accuracy, high efficiency, simple operation, low cost and good application prospect.

The above contents of the present invention will be further described as below in details by specific implementations in the form of embodiments, and the scope of the subject of the present invention should not be interpreted as being limited thereto. All technologies realized on the basis of the contents of the present invention shall fall into the scope of the present invention.

### BRIEF DESCRIPTION OF THE DRAWINGS

FIG. 1 is a process diagram of synthesizing compounds by combinatorial chemistry method according to the present invention, wherein “H” represents synthetic building blocks; “initial” represents an initial sequence; “B” represents tag sequences which specifically tag the synthetic building blocks, a number following it represents a correspondence between the both, for example, B1 specifically tags H1; “terminal” represents a terminal sequence; the left column represents the resulting products from reaction steps which are consistent to the reaction steps in Embodiment 1; for the synthetic building blocks, an order from right to left merely represents an order of adding the synthetic building blocks; and for the initial sequence, the tag sequences and the terminal sequence, an order from left to right represents a structure of the finally obtained single-stranded DNA sequence;

FIG. 2 is an electrophoresis image of a chemical library and a trypsin inhibitor obtained by screening according to the present invention;

FIG. 3 is a column chart of the results of sequencing, where the columns are in one-to-one DNA tags correspondence to compounds, and the height of the columns is related to the bonding force of the compounds to a target;

FIG. 4 is an IC50 curve of a trypsin inhibitor according to the present invention; and

FIG. 5 is an IC50 curve of the trypsin inhibitor according to the present invention.

### DETAILED DESCRIPTION OF THE EMBODIMENTS

The specific implementations will be stated as below in the form of embodiments, and the contents of the present invention will be further described in details. However, the scope of the subject of the present invention should not be interpreted as being limited thereto. All technologies realized on the basis of the contents of the present invention shall fall into the scope of the present invention.

### Embodiment 1

A method for synthesizing and screening lead compounds

1. Preparation method

1)Preparation of synthetic building blocks and single-stranded DNA fragments i synthetic building blocks and (i+2) single-stranded DNA fragments are prepared, where the (i+2) single-stranded DNA fragments include i tag sequences, a start sequence and a terminal sequence, and the i tag sequences specifically tag the i synthetic building blocks, respectively, where i=1, 2, 3 . . . n.

Poly-adenosine may be linked to the start sequence for convenient separation and purification. Cytidines may be linked to the tag sequences in order to improve the ligation efficiency of the subsequent single-stranded DNA fragments using RNA ligase

TABLE 1
Single-stranded DNA fragments

| Name of | | | |
|---|---|---|---|
| sequence | | Sequence of single-stranded DNA | Synthetic building blocks |
| Start | | 5′-PO3-AGATCTGATGGCGCGAG | |
| sequence | | GGAAAAAAAAAAAA-3′-PO4 | |
| Tag sequences | 1 | TCAGGCAGAc | 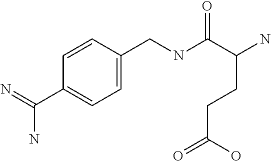 [SMILES: N=C(N)c1ccc(CNC(=O)C(N)CCC(=O)O)cc1] |
| | 2 | AGCATTTCAc | 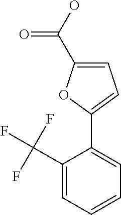 [SMILES: O=C(O)c1ccc(-c2ccccc2C(F)(F)F)o1] |
| | 3 | CGACTTAGCc | 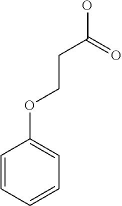 [SMILES: O=C(O)CCOc1ccccc1] |
| | 4 | GGAGTTCAAc | 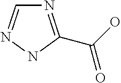 |
| | 5 | CTACGAGAAc | 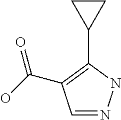 [SMILES: O=C(O)c1cn[nH]c1C1CC1] |
| | 6 | TAGGCGTTAc | 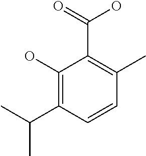 |
| | 7 | CGTTCTAATc | 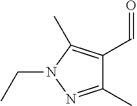 |
| | 8 | GGGAACGCGc | 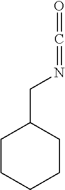 |
| | 9 | TTGTAGATCc | 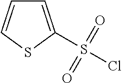 |
| | . | . | . |
| | . | . | . |
| | . | . | . |
| | n | TCTATGGGTc | 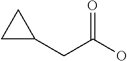 |

| | |
|---|---|
| Terminal | GGAGCTTGTGAATTCTGGc |
| sequence | |

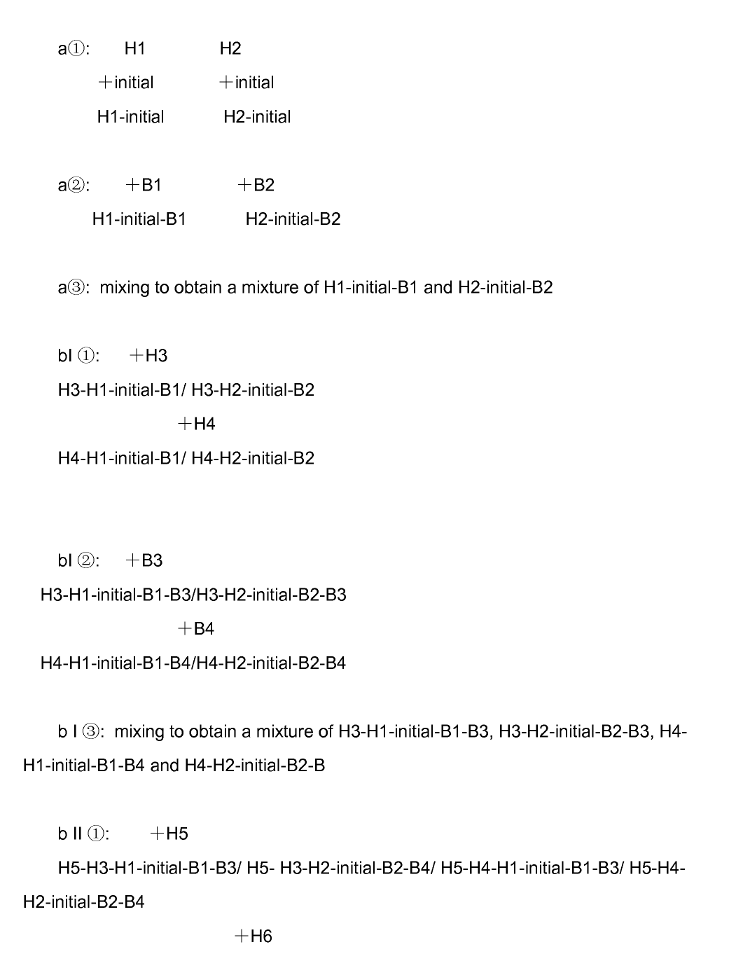

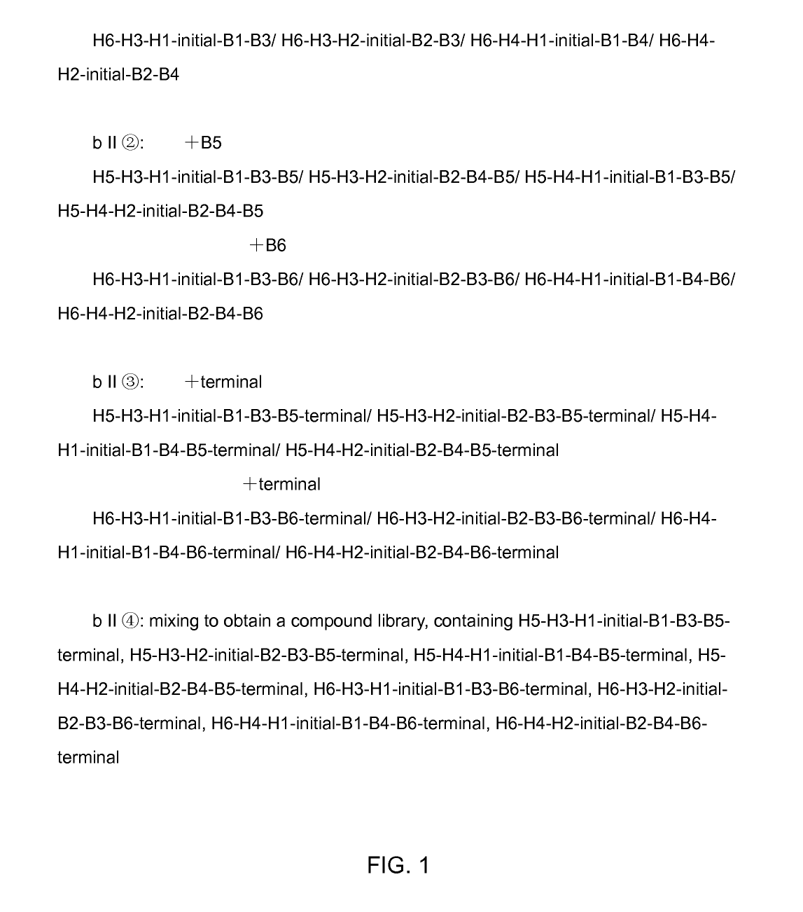

(2) Synthesis: As shown in FIG. 1, a compound library is synthesized by combinatorial chemistry method:

a: preparation of initial synthetic building blocks: selecting 1 to i synthetic building blocks, linking one end of the start sequence to a synthetic building block and the other end of the start sequence in series to a specific tag sequence of the synthetic building block, to obtain 1 to i initial synthetic building blocks tagged with single-stranded DNA with a free end, where, for example, i=2;

\textcircled{1} initial synthetic building blocks are linked to the start sequence:

the start sequence is aminated, synthetic building blocks 1 and 2 are carboxylated, sulfhydrylized or alkynylated; then, the activated synthetic building blocks 1 and 2 are reacted with the activated start sequence to obtain initial synthetic building blocks linked to the start sequence;

\textcircled{2} the tag sequences of the synthetic building blocks 1 and 2 are linked to the start sequence, respectively (as this linking method, in addition to the following methods, other linking methods for single-stranded DNA may be used):

5′-end of the single-stranded DNA is phosphorylated using polynucleotide kinase and then linked using RNA ligase;

\textcircled{3} the initial synthetic building blocks are mixed to obtain a mixture of initial synthetic building blocks.

b: Based on the initial synthetic building blocks obtained in step a, compounds are synthesized in a manner of linear combination reaction, wherein, during synthesis, once a new synthetic building block is added, a specific tag sequence of this new synthetic building block is linked in series to the free end of the single-stranded DNA linked to the initial synthetic building blocks such that the single-stranded DNA is gradually lengthened; at the end of synthesis, the terminal sequence is linked in series to the free end of the single-stranded DNA to obtain a single-stranded DNA-encoded compound library; for example, a three-step linear combination reaction.

I:

\textcircled{1} synthesis (in addition to the following synthesis methods, other chemical synthesis methods may be used): synthetic building block 3-4 are placed into two miniature reaction vessels, then separately mixed with the mixture of initial synthetic building blocks obtained in step a, and synthesized by mixed splitting, parallel synthesis, multi-component liquid-phase synthesis or functional group conversion;

\textcircled{2} adding the tag sequences: the same as step \textcircled{2} of step a; and

\textcircled{3} mixing to obtain a mixture.

II:

\textcircled{1} synthesis (in addition to the following synthesis methods, other chemical synthesis methods may be used): synthetic building block 5-6 are placed into two miniature reaction vessels, then separately mixed with the mixture obtained in step b, and synthesized by mixed splitting, parallel synthesis, multi-component liquid-phase synthesis or functional group conversion;

\textcircled{2} adding the tag sequences: the same as step \textcircled{2} of step a;

\textcircled{3} adding the terminal sequence: the same as step \textcircled{2} of step a; and

\textcircled{4} mixing to obtain a library of single-stranded DNA-encoded compounds.

(3) Screening: the DNA-encoded compound library is screened:

Through a chromatographic separation and screening method based on a receptor-ligand specific reaction, the DNA-encoded compound library is screened with biological target molecules.

Elution is carried out in the chromatographic column to separate and remove DNA-encoded compounds which are not bonded with the biological target molecules to obtain DNA-encoded compounds bonded with the biological target molecules.

(4) Sequencing:

DNA on the DNA-encoded compounds screened in step (3) is sequenced, so that the synthetic building blocks and reaction mechanisms of these compounds may be determined according to the DNA sequence.

### Embodiment 2

Synthesizing and screening trypsin ligand using the method provided by the present invention

1. Materials and reagents

T4 PNK (500U NEB-M0201V), T4 RNA ligase 1(NEB-M0204S), Cartridges (PCR purification Kit (cat.no 28104, Nucleotides removal Kit cat.no 28306) purchased from Qiagen (Hilden, Germany), and dNTPs (0.5 mM, NEB, cat.no 89009).

The single-stranded DNA fragments shown in Table 1 are synthesized by Genscript and Biosune.

2. Preparation method

(1) Preparation of single-stranded DNA fragments

The synthetic building blocks totally used in this embodiment and their coding sequences are given.

54 synthetic building blocks and 56 single-stranded DNA fragments are used. The 57 single-stranded DNA fragments include 55 tag sequences, one start sequence and one terminal sequence.

Cytidines may be linked to the following tag sequences in order to improve the ligation efficiency of the subsequent single-stranded DNA fragments using T4 RNA ligase.

TABLE 2
Single-stranded DNA fragments

| Name of | | | |
|---|---|---|---|
| sequence | | Sequence of single-stranded DNA | Synthetic building blocks |
| Start | | 5′-PO3-AGATCTGATGGCGCGAG | |
| sequence | | GGAAAAAAAAAAAA-3′-PO4 | |
| Tag sequences | 1 | TGCCCAAGGc | 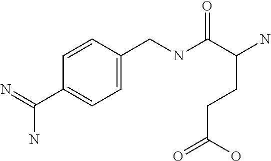 [SMILES: N=C(N)c1ccc(CNC(=O)C(N)CCC(=O)O)cc1] |
| | 2 | CGTCTCGATc | 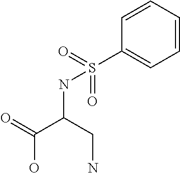 [SMILES: NCC(NS(=O)(=O)c1ccccc1)C(=O)O] |
| | 3 | TGCGCCGAGc | 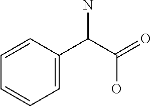 [SMILES: NC(C(=O)O)c1ccccc1] |
| | 4 | ATGGATTTAc | 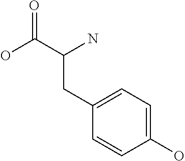 [SMILES: NC(Cc1ccc(O)cc1)C(=O)O] |
| | 5 | CATGTTTACc | 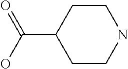 |
| | 6 | GTAACATTAc | 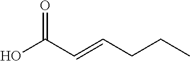 |
| | 7 | GGAGTTCAAc | 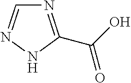 |
| | 8 | CTTTGTACTc | 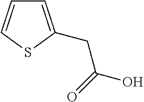 |
| | 9 | ACTACCGTGc | 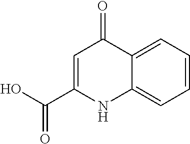 [SMILES: O=C(O)c1cc(=O)c2ccccc2[nH]1] |
| | 10 | ATGAATAAGc | 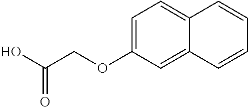 [SMILES: O=C(O)COc1ccc2ccccc2c1] |
| | 11 | AAGAATTTAc | 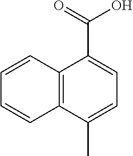 |
| | 12 | CACCATTATc | 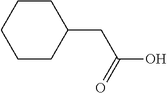 |
| | 13 | AGAGGGAAGc | 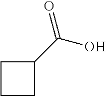 |
| | 14 | GTCGGTGGAc | 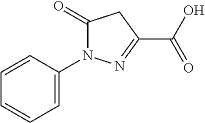 [SMILES: O=C(O)C1=NN(c2ccccc2)C(=O)C1] |
| | 15 | GGGATGATGc | 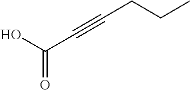 |
| | 16 | AAAACAGGGc | 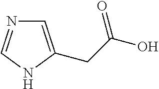 |
| | 17 | ATTGATGATc | 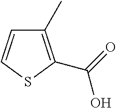 |
| | 18 | GCACCCTCAc | 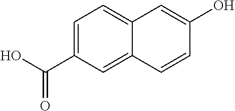 [SMILES: O=C(O)c1ccc2cc(O)ccc2c1] |
| | 19 | TGGTAAAGGc | 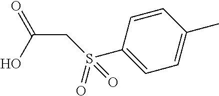 |
| | 20 | CACTTAGCGc | 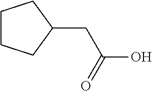 |
| | 21 | AATGTAGAAc | 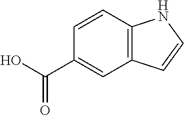 [SMILES: O=C(O)c1ccc2[nH]ccc2c1] |
| | 22 | CGTGCTCCAc | 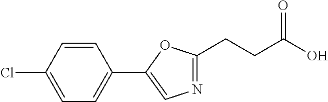 [SMILES: O=C(O)CCc1ncc(-c2ccc(Cl)cc2)o1] |
| | 23 | ACGCGCATAc | 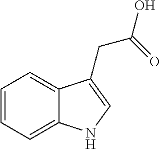 [SMILES: O=C(O)Cc1c[nH]c2ccccc12] |
| | 24 | TGGCGCACTc | 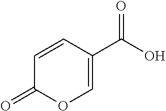 |
| | 25 | CTACGAGAAc | 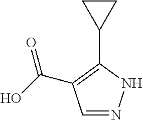 [SMILES: O=C(O)c1cn[nH]c1C1CC1] |
| | 26 | CTGTGACCTc | 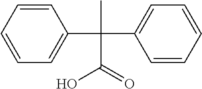 [SMILES: CC(C(=O)O)(c1ccccc1)c1ccccc1] |
| | 27 | GAAGAAGACc | 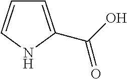 |
| | 28 | TAAATAGTTc | 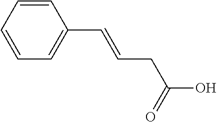 [SMILES: O=C(O)C/C=C/c1ccccc1] |
| | 29 | TCCTAGCTTc | 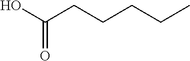 |
| | 30 | TCCCTACCAc | 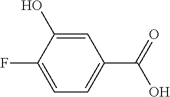 [SMILES: O=C(O)c1ccc(F)c(O)c1] |
| | 31 | AGGTCCCGAc | 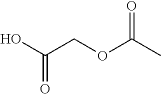 |
| | 32 | TAAGGATGAc | 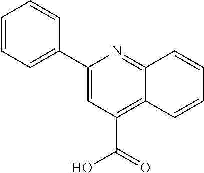 [SMILES: O=C(O)c1cc(-c2ccccc2)nc2ccccc12] |
| | 33 | TTGCTCTTAc | 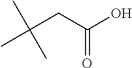 |
| | 34 | TCCAACGACc | 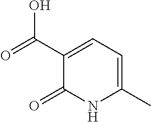 |
| | 35 | TACATCTTCc | 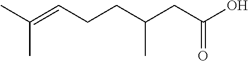 |
| | 36 | GTTGCAGGTc | 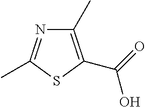 |
| | 37 | CCGGGCTTGc | 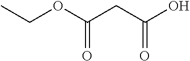 |
| | 38 | GGCGATAGAc | 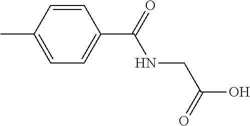 |
| | 39 | CTTCTGACCc | 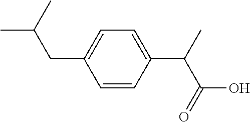 |
| | 40 | GTGCGACGCc | 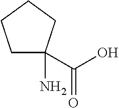 |
| | 41 | AGTAAACGAc | 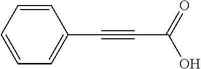 [SMILES: O=C(O)C#Cc1ccccc1] |
| | 42 | CATCGCCCGc | 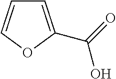 |
| | 43 | AAACCGACTc | 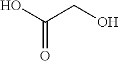 |
| | 44 | CAACCATGGc | 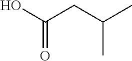 |
| | 45 | TCTCCATTGc | 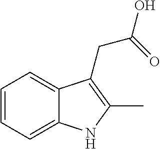 |
| | 46 | AGCATTTCAc | 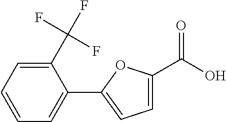 [SMILES: O=C(O)c1ccc(-c2ccccc2C(F)(F)F)o1] |
| | 47 | TACGCAAACc | 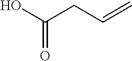 |
| | 48 | ATAACCTGGc | 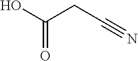 |
| | 49 | CGAAGCGTTc | 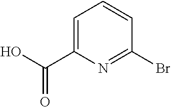 |
| | 50 | TAGGCGTTAc | 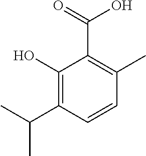 |
| | 51 | TGCCAACATc | 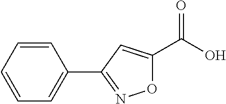 [SMILES: O=C(O)c1cc(-c2ccccc2)no1] |
| | 52 | CGACTTAGCc | 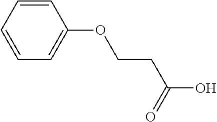 [SMILES: O=C(O)CCOc1ccccc1] |
| | 53 | GTATGAAAAc | 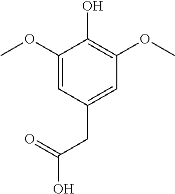 |
| | 54 | TTGGCAGGGc | 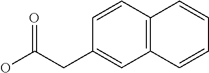 [SMILES: O=C(O)Cc1ccc2ccccc2c1] |
| | 55 | TAGATATTGc | 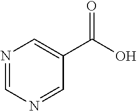 |

| | |
|---|---|
| Terminal | GGAGCTTGTGAATTCTGGc |
| sequence | |

(2) Synthesis:

a: Preparation of an initial synthetic building block: selecting a synthetic building block, linking one end of the start sequence to a synthetic building block and the other end of the start sequence to a specific tag sequence of the synthetic building block in series, to obtain an initial synthetic building block tagged with single-stranded DNA with a free end;

\textcircled{1} the initial synthetic building block is linked to the start sequence:

the start sequence is aminated, synthetic building block 1 is carboxylated, sulfhydrylized or alkynylated; then, the activated synthetic building block 1 is reacted with the activated start sequence to obtain the initial synthetic building block linked to the start sequence.

The total volume of the reaction mixture is 150 μL, and the solvents are water and dimethylsulfoxide at a volume ratio of 3:7 and contain a triethylamine hydrochloride buffer system (pH 10.0, 80 mM), wherein the concentration of the synthetic building block 1 is 30 mM, the concentration of 1-ethyl-3-(3-dimethylaminopropyl) carbodiimide hydrochloride (EDCI) (an activating agent) is 4 mM and the concentration of 2-sulfo-N-hydroxyl succinimide (an activating agent) is 10 mM, the concentration of the start sequence is 20 M, and the reaction is performed at the room temperature for 1 h.

\textcircled{2} The tag sequence of the synthetic building block 1 is linked to the start sequence (as this linking method, in addition to the following methods, other linking methods for single-stranded DNA may be used):

5′-end of the single-stranded DNA is phosphorylated using polynucleotide kinase and then linked using RNA ligase.

Linking: the treated start sequence in step \textcircled{1} and the tag sequence 1 are ready for use. 15 μL of the reaction mixture contains 225 pmol of the start sequence, 25 pmol of the tag sequence 1, 50 units of the T4 RNA ligase and a buffer solution for the linking reaction. The mixture is incubated at 25° C. for 1.5 h and then heated at 70° C. for 20 min, and the T4 RNA ligase is denatured. Subsequently, T4 polynucleotide kinase and 1 nm of ATP are added into the mixture, then reacted for 10 cycles and incubated at 75° C. for 20 min to denature the extra polynucleotide kinase.

Purification: the resulting product is placed in a 2× loading buffer solution. The buffer solution contains 40 mM of Tris-HCL (pH7.6), 1M of NaCL and 1 mM of EDTA.

The obtained mixture is purified by the following steps: the reaction liquid is put in a Qiagen Cartridge column, then suspended with 1× loading buffer solution, centrifuged at 100 rmp for 1 min, filtered by siliconized glass wool, then successively washed with 1× loading buffer solution, 0.5 M of NaCl solution and 80% of ethyl alcohol, eluted with 20 μL of PE eluant and dried in vacuum.

b: Based on the initial synthetic building block obtained in step a, compounds are synthesized in a manner of three-step linear combination reaction, wherein, during synthesis, once a new synthetic building block is added, a specific tag sequence of this new synthetic building block is linked in series to the free 3′-end of the single-stranded DNA linked to the initial synthetic building blocks such that the single-stranded DNA is gradually lengthened; at the end of synthesis, the terminal sequence is linked in series to the free end of the single-stranded DNA to obtain a single-stranded DNA-encoded compound library.

First batch of synthetic building blocks, i.e., the initial building block (1): synthetic building block 1;

Second batch of synthetic building blocks (5): synthetic building blocks 2-6; and,

Third batch of synthetic building blocks (49): synthetic building blocks 7-55.

I:

\textcircled{1} Synthesis

Synthetic building blocks 2-6 are placed into five miniature reaction vessels, then separately mixed with the initial synthetic building block obtained in step a, and synthesized by mixed splitting, parallel synthesis, multi-component liquid-phase synthesis or functional group conversion;

These synthetic building blocks are placed into five miniature reaction vessels and then reacted with the initial synthetic building block obtained in step a, respectively. Taking the synthetic building block 2 for example, the reaction conditions are as follows: in 150 μL of reaction mixture, the solvents are water and dimethylsulfoxide at a volume ratio of 3:7 and contain a triethylamine hydrochloride buffer system (pH 9.0, 80 mM), wherein the concentration of the synthetic building block 1 is 30 mM, the concentration of 1-ethyl-3-(3-dimethylaminopropyl) carbodiimide hydrochloride (EDCI) (an activating agent) is 4 mM and the concentration of 2-sulfo-N-hydroxyl succinimide (an activating agent) is 10 mM, the concentration of the synthetic building block 2 is 1.5 M, and the reaction is performed at the room temperature for 15 h.

\textcircled{2} Adding the tag sequences of the synthetic building blocks 2-6: the same as step \textcircled{2} of step a.

\textcircled{3} Mixing to obtain a mixture.

II:

\textcircled{1} Synthesis

Synthetic building blocks 7-55 are placed into 49 miniature reaction vessels, then separately mixed with the initial synthetic building block obtained in step a, and synthesized by mixed splitting, parallel synthesis, multi-component liquid-phase synthesis or functional group conversion;

These synthetic building blocks are placed into 49 miniature reaction vessels and then reacted with the initial synthetic building block obtained in step a, respectively. Taking the synthetic building block 2 for example, the reaction conditions are as follows: in 150 μL of reaction mixture, the solvents are water and dimethylsulfoxide at a volume ratio of 3:7 and contains a triethylamine hydrochloride buffer system (pH 9.0, 80 mM), wherein the concentration of the synthetic building block 1 is 30 mM, the concentration of 1-ethyl-3-(3-dimethylaminopropyl) carbodiimide hydrochloride (EDCI) (an activating agent) is 4 mM and the concentration of 2-sulfo-N-hydroxyl succinimide (an activating agent) is 10 mM, the concentration of the synthetic building block 2 is 1.5 M, and the reaction is performed at the room temperature for 15 h.

\textcircled{2} Adding tag sequences: the same as step \textcircled{2} of step a.

\textcircled{3} Adding the terminal sequence: the same as step \textcircled{2} of step a.

\textcircled{4} Mixing to obtain a library of single-stranded DNA-encoded compounds.

(3) Screening: the DNA-encoded compound library is screened:

Through a chromatographic and separation screening method based on a receptor-ligand specific reaction, the DNA-encoded compound library is screened with biological target molecules.

\textcircled{1} CNBr resin activation

1) Sepharose 4B resin is activated with 0.1033 g of CBNr, then divided into two branches, and stood in 4 ml of 1 mM hydrogen chloride solution (pH3.0).

2) Washing with 1 mM of hydrochloric acid (pH=3.0) washing liquid is performed for 15 min.

3) 4 mg of trypsin is dissolved in 0.5 ml of coupling buffer solution (0.1 M of sodium hydrogen carbonate, 0.5 M of sodium chloride, pH=8.3).

4) The mixture is slightly shocked up and down for 1 h and then incubated over night at the room temperature or 4° C.

5) Excessive protein is removed with 4 ML of coupling solution.

6) The resin is transferred into 4 mL of 0.1 M Tris-HCl solution (pH 8.0) and incubated for 2h.

7) The resin is washed with washing buffer solutions 1 and 2 for three times (washing solution 1: 0.1 M of acetic acid, 0.5 M of NaCl, pH 4.0; washing solution 2: 0.1 M of Tris-HCl, 0.5 M pf NaCl, pH 8.0).

8) The resin is centrifuged at 6000 r/min for 10 min.

\textcircled{2} Solidification of trypsin on the activated CNBr resin

1) 100 mg of the activated CNBr resin is put into 4 ml of 1 mM hydrochloric acid for incubation;

2) washing with 8 mL of 1 mM hydrochloric acid (pH=3.0) is performed;

3) 0.004 mg/ml, 0.02 mg/ml, 0.1 mg/ml, 0.5 mg/ml and 2.5 mg/ml of trypsin solutions are mixed with five parts of CNBr resin and then incubated at 4° C. for 5 h, respectively;

4) the resin is washed with 0.1 M of Tris hydrochloric acid and 0.5 M of sodium chloride (pH=8.3);

5) the resin is washed with 0.1 M of sodium acetate and 0.5 M of sodium chloride (pH=4.0);

6) steps 4 and 5 are repeated for alternately washing for at least three cycles; and

7) the resin solidifying trypsin is stored in PBS buffer solution (pN=7.4) at 4° C.

\textcircled{3} Affinity screening of a trypsin compound library

1) The library of single-stranded DNA-encoded compounds obtained in step (2) is mixed with PBS buffer solution at a volume ratio of 1:15 (17 μL:255 μL);

2) 50 μL of the library sample is added into bovine pancreas trypsin/CNBr resin slurry (2.5, 0.5, 0.1, 0.02, 0.004 and 0 mg/mL);

3) 0.3 mg/mL of herring sperm DNA solution is prepared using PBS buffer solution;

4) the herring sperm DNA solution obtained in step 3) and the bovine pancreas trypsin/CNBr resin slurry obtained in step 2) are incubated at 25° C. for 1 h;

5) the mixture obtained in step 4) is transferred to a 2 ml Spin column, and supernatant is removed;

6) the resin is washed with 200 μL of PBS buffer solution for 4 times; and

7) the washed slurry is added with 100 μL of sterile water and then screened to obtain a trypsin ligand sample.

Identification: electrophoresis detection is performed to the single-stranded DNA-encoded compound library obtained in step (2) and the trypsin affine sample screened in step (3).

The result of detection is as shown in FIG. 2. A target band is obtained by screening using bovine pancreas trypsin/CNBr resin slurry and a blank band is obtained in the negative control, so that it is indicated that purified trypsin affine sample is obtained by screening in the present invention.

(4) Sequencing:

DNA on the DNA-encoded compounds screened in step (3) is sequenced, so that the synthetic building blocks and reaction mechanisms of these compounds may be determined according to the DNA sequence.

The sample screened in step (3) is subjected to a polymerase chain reaction (PCR), the oligonucleotide codes of the encoded compounds are subjected to PCR amplification (a total volume of 50 μL, 30 cycles each for 1 min at 94° C., for 1 min at 55° C. and for 40 s at 72° C.), and 5 μL of trypsin 245 library (a concentration of 100 fM) is used as a template.

An Illumina Hiseq2500 high throughput sequencing platform is employed, and the sequencing flow is as follows:

1) the PCR-amplified screened oligonucleotide library is purified by a MAG-PCR-CL-250 kit produced by Axygen, and a quality test report is provided;

2) nuclear acid is quantified by a Picogreen kit produced by Illumina to obtain the concentration of nuclear acids of the sample, ready for a next step of sequencing the library;

3) a Hiseq2000 specific sequencing adaptor is linked to the 5′-end and 3′-end of a sequenced sample by a chip-seq DNA sample kit produced by Illumina, and then fixed on a chip chip-seq plate of a Hiseq2500 sequencer for a next step of bridge amplification;

4) the bridge amplification of a nuclear acid sample is performed by a kit Truseq PE Cluster Kit v3-cBot-HS, and a nuclear acid cluster sufficient for sequencing is obtained on each chip-seq lane;

5) the appearance order and frequency of each base are read from the sequencing adaptor by a labeled dNTP of Truseq SBS Kit v3-HS (200 cycles) of a laser imaging system of Hiseq 2500, and the bases of the nuclear acid sample are tested; and

6) data is taken out and then processed.

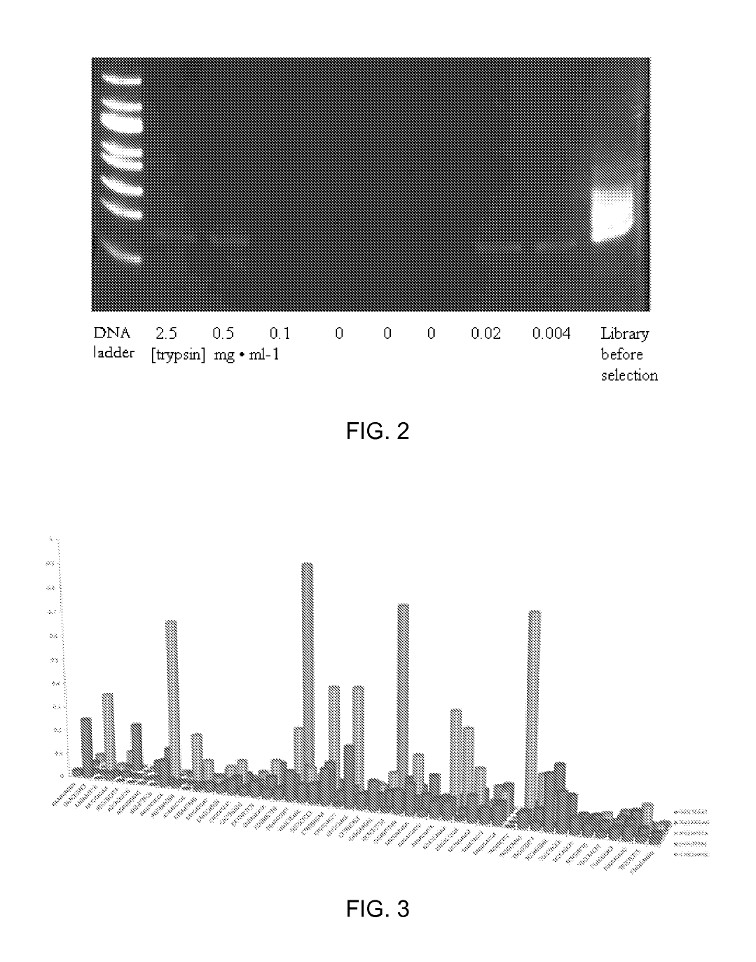

The result of sequencing is as shown in FIG. 3, and the sequence is shown by SEQ ID NO.1: TCAGGCAGAGGCGATAGAGGCGATAGA. With reference to Table 2, the structure of the screened trypsin ligand may be determined as follows:

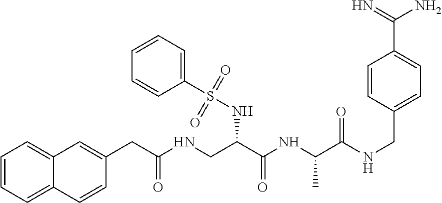 [SMILES: C[C@H](NC(=O)[C@H](CNC(=O)Cc1ccc2ccccc2c1)NS(=O)(=O)c1ccccc1)C(=O)NCc1ccc(C(=N)N)cc1]

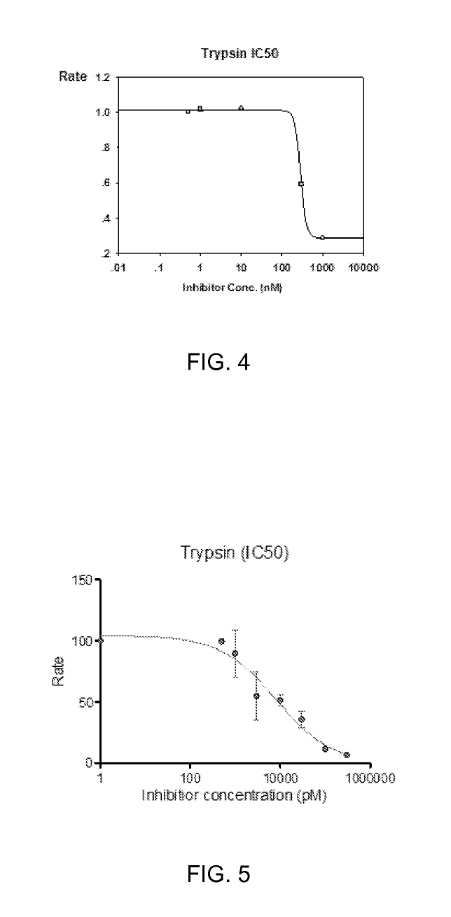

According to the structural formula, the compound is tested at the end of synthesis. It is determined by tests that the compound is a trypsin inhibitor, the enzyme inhibition activity of which is as shown in FIGS. 4-5, where IC50 is 8.1±2.1 nM. Thus, it is indicated that the screened compound is definitely a trypsin ligand.

The experimental results show that the present invention establishes a chemical library containing 245 compounds, and obtains by screening a trypsin ligand having a trypsin inhibition activity. Therefore, it is indicated that the method provided by the present invention may effectively synthesize and screen lead compounds.

### Embodiment 3

A kit for synthesizing and screening lead compounds

Compositions of the kit provided by the present invention (dosage of N synthetic building blocks for synthesis)

1) i synthetic building blocks and (i+2) single-stranded DNA fragments, where the single-stranded DNA fragments include a start sequence, a terminal sequence and i tag sequences, and the i tag sequences specifically tag the i synthetic building blocks, respectively, where i=1, 2, 3 . . . n;

TABLE 3
Synthetic building blocks and single-stranded DNA fragments of different
sequences

| Name of | | Sequence of single-stranded DNA, | Synthetic building block, |
|---|---|---|---|
| sequence | | 1.5 M | 30 mM |
| Start | | 5′-PO3-AGATCTGATGGCGCGAG | |
| sequence | | GGAAAAAAAAAAAA-3′-PO4 | |
| Tag sequences | 1 | TCAGGCAGAc | 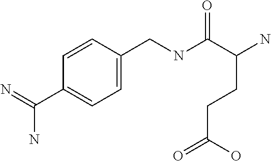 [SMILES: N=C(N)c1ccc(CNC(=O)C(N)CCC(=O)O)cc1] |
| | 2 | AGCATTTCAc | 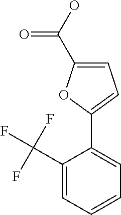 [SMILES: O=C(O)c1ccc(-c2ccccc2C(F)(F)F)o1] |
| | 3 | CGACTTAGCc | 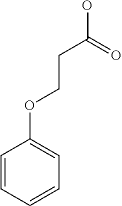 [SMILES: O=C(O)CCOc1ccccc1] |
| | 4 | GGAGTTCAAc | 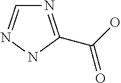 |
| | 5 | CTACGAGAAc | 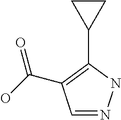 [SMILES: O=C(O)c1cn[nH]c1C1CC1] |
| | 6 | TAGGCGTTAc | 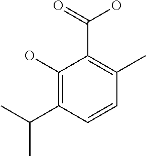 |
| | 7 | CGTTCTAATc | 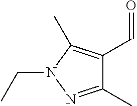 |
| | 8 | GGGAACGCGc | 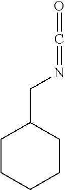 |
| | 9 | TTGTAGATCc | 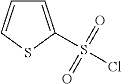 |
| | . | . | . |
| | . | . | . |
| | . | . | . |
| | n | TCTATGGGTc | 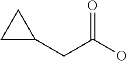 |

| | |
|---|---|
| Terminal | GGAGCTTGTGAATTCTGGc |
| sequence | |

2) a reagent for linking the start sequence-initial synthetic building blocks, a reagent for combinatorial chemistry method and a reagent for linking the single-stranded DNA fragments;

TABLE 4
Reagent for linking the start sequence to the synthetic building blocks

| Name of reagent | Dosage |
|---|---|
| Triethylamine hydrochloride buffer solution, pH 10.0 | (800 mM, 15 μL) |
| 1-ethyl-3-(3-dimethylaminopropy) carbodiimide | (40 mM, 15 μL) |
| 2-sulfo-N-hydroxyl succinimide | (100 mM, 15 μL) |

TABLE 5
Reagent for combinatorial chemistry method

| Name of reagent | Dosage |
|---|---|
| Triethylamine hydrochloride buffer solution, pH 9.0 | (800 mM, 15 μL) |
| 1-ethyl-3-(3-dimethylaminopropy) carbodiimide | (40 mM, 15 μL) |
| 2-sulfo-N-hydroxyl succinimide | (100 mM, 15 μL) |

TABLE 6
Reagent for linking DNA fragments

| Name of reagent | Dosage |
|---|---|
| T4 PNK (10 U/μl) | N × 10 μL |
| 10 × T4 RNA ligase buffer | N × 10 μL |
| dd H2O | N × 77.4 μL |
| T4 RNA ligase (10 U/μl) | N × 10 μL |
| 10 × T4 RNA ligase buffer | N × 2.5 μL |
| ATP(10 mM) | N × 0.1 μL |

3) a reagent for screening compounds;

TABLE 7
Reagent for screening compounds

| Name of reagent | Dosage |
|---|---|
| Biological target | Trypsin concentration |
| molecules | 2.5 mg/ml, 0.5 mg/ml, 0.1 mg/ml, |
| | 0.02 mg/ml, 0.004 mg/ml |
| CBNr Sepharose 4B | 100 mg/batch |
| (GE 17-0340-01) | |
| Enzyme immobilization | 0.1M NaHCO3, 0.5M NaCl, pH 8.3) |
| buffer solution | |
| Washing buffer solution | 0.1M acetic acid, 0.5M NaCl, pH4.0 |
| 1 | |
| Washing buffer solution | 0.1M Tris-HCI, 0.5M NaCl, pH8.0 |
| 2 | |
| PBS buffer solution | 20 mM NaH2PO4, 30 mM Na2HPO4, |
| | 100 mM NaCl [pH 7.4]) |
| Herring sperm DNA | 0.3 mg/mL, 100 uL/sample |

4) a reagent for DNA sequencing.

TABLE 8
Reagent for DNA sequencing

| Purpose | Name of reagent |
|---|---|
| PCR purification | MAG-PCR-CL-250 |
| Nuclear acid | Picogreen kit |
| quantification | |
| Library | chip-seq DNA sample kit |
| construction | |
| Bridge | Truseq PE Cluster Kit v3-cBot-HS |
| amplification | |
| Online sequencing | Truseq SBS Kit v3-HS (200cycles) |

The kit provided by the present invention is used according to the method provided by Embodiment 1 of the present invention and may be used for rapidly synthesizing and screening lead compounds.

In conclusion, compared with using double-stranded DNA to tag synthetic building blocks in the prior art, the present invention uses single-stranded DNA to tag synthetic building blocks. As the single-stranded DNA will not be complementary and difficult to be cross-linked during linking and has stable structure, the PCR amplification and sequencing of the single-stranded DNA are more convenient and rapid when compared with the case of using the double-stranded DNA. Therefore, the method provided by the present invention may contain multiple linear combination reaction steps, the synthesized compound library has high diversity and large capacity, and it is easy to synthesize target compounds. By sequencing, the synthetic building blocks, the reaction mechanisms and the chemical structures may be determined. Therefore, the method provided by the present invention has high accuracy, high efficiency, simple operation, low cost and good application prospect.

1. A method for synthesizing and screening lead compounds, comprising the following steps of:
(1) Preparing raw materials, i.e., i synthetic building blocks and (i+2) single-stranded DNA fragments, where the (i+2) single-stranded DNA fragments comprise i tag sequences, a start sequence and a terminal sequence, and the i tag sequences specifically tag the i synthetic building blocks, respectively, where i−1, 2, 3 . . . n;
(2) Synthesizing a compound library by combinatorial chemistry method:
a, preparing initial synthetic building blocks: selecting 1 to i synthetic building blocks, linking one end of the start sequence to a synthetic building block and the other end of the start sequence in series to a specific tag sequence of the synthetic building block, to obtain 1 to i initial synthetic building blocks tagged with single-stranded DNA with a free end;
b, synthesizing compounds by reacting the initial synthetic building blocks obtained in step a and the 1 to i synthetic building blocks in a manner of linear combination, wherein, during synthesis, once a new synthetic building block is added, a specific tag sequence of this new synthetic building block is linked in series to the free end of the single-stranded DNA linked to the initial synthetic building blocks such that the single-stranded DNA is gradually lengthened; at the end of synthesis, the terminal sequence is linked in series to the free end of the single-stranded DNA to obtain a single-stranded DNA-encoded compound library;
(3) Screening: screening the DNA-encoded compound library to select target compounds; and
(4) Sequencing: sequencing the DNA of the target compounds screened in step (3), and determining synthetic building blocks and reaction mechanisms for the target compounds.

2. The method according to claim 1, characterized in that the start sequence in step (1) comprises poly-adenosine comprising 12-20 adenosines.

3. (canceled)

4. The method according to claim 1, characterized in that the length of the tag sequences in step (1) is not less than 6 bp.

5. The method according to claim 4, characterized in that the length of the tag sequences is 9 bp.

6. The method according to claim 1, characterized in that, a ribonucleotide is linked to the 3′-end of the tag sequences in step (1), and the ribonucleotide is cytidine.

7. (canceled)

8. The method according to claim 1, characterized in that, in step (2), a method for linking the start sequence to the initial synthetic building blocks in step a is as follows:
Performing amination to the start sequence, performing carboxylation, sulfhydrylization or alkynylation to the initial synthetic building blocks, and reacting the start sequence with the initial synthetic building blocks.

9. The method according to claim 1, characterized in that, in step (2), during synthesis, the pH is 8-12 and the temperature is 0-30° C.

10. The method according to claim 1, characterized in that, in step (2), a method for linking the start sequence to the tag sequences, linking the tag sequences or linking the tag sequences to the terminal sequence is as follows: phosphorylating the 5′-end of the single-stranded DNA with polynucleotide kinase and then linking using RNA ligase.

11. The method according to claim 10, characterized in that the polynucleotide kinase is T4 polynucleotide kinase, and the RNA ligase is T4 RNA ligase.

12. The method according to claim 1, characterized in that the screening method in step (3) is one based on a receptor-ligand specific reaction.

13. A kit for synthesizing and screening lead compounds, comprising the following components:
1) i synthetic building blocks and (i+2) single-stranded DNA fragments, where the (i+2) single-stranded DNA fragments comprise i tag sequences, a start sequence and a terminal sequence, and the i tag sequences specifically tag the i synthetic building blocks, respectively, where i=1, 2, 3 . . . n;
2) a reagent for linking the start sequence-initial synthetic building blocks, a reagent for combinatorial chemistry method and a reagent for linking the single-stranded DNA fragments;
3) a reagent for screening compounds; and
4) a reagent for DNA sequencing.

14. The kit according to claim 13, characterized in that the start sequence in component 1) comprises poly-adenosine comprising 12 to 20 adenosines.

15. (canceled)

16. The kit according to claim 13, characterized in that the length of the tag sequences in component 1) is not less than 6 bp.

17. The kit according to claim 16, characterized in that the length of the tag sequences in component 1) is 9 bp.

18. The kit according to claim 13, characterized in that, a ribonucleotide is linked to the 3′-end of the tag sequences in component 1), and the ribonucleotide is cytidine.

19. (canceled)

20. The kit according to claim 13, characterized in that, the reagent for linking the start sequence in component 2) to the synthetic building blocks comprises a reagent for amination of the start sequence, and a reagent for carboxylation, sulfhydrylization or alkynylation of the synthetic building blocks.

21. The kit according to claim 13, characterized in that, the reagent for linking single-stranded DNA fragments in component 2) comprises polynucleotide kinase and RNA ligase.

22. The kit according to claim 21, characterized in that the polynucleotide kinase is T4 polynucleotide kinase, and the RNA ligase is T4 RNA ligase.

23. A combinatorial chemistry library, synthesized by combinatorial chemistry method using synthetic building blocks as raw materials, wherein a fragment of a single-stranded DNA sequence is tagged for each compound; and the single-stranded DNA sequence has a following structure: a start sequence-i tag sequences-a terminal sequence, the i tag sequences specifically tag i synthetic building blocks used during the combinatorial chemistry synthesis, and the order of the i tag sequences is the same as an order of adding the synthetic building blocks during the combinatorial chemistry synthesis.

24. The combinatorial chemistry library according to claim 23, characterized in that the length of the tag sequences is not less than 6 bp.

25. The combinatorial chemistry library according to claim 24, characterized in that the length of the tag sequences is 9 bp.


SEQUENCE LISTING (68 sequences)

SEQ ID NO: 1 - DNA, 27 bp, Artificial Sequence
  Synthetic
tcaggcagaggcgatagaggcgataga

SEQ ID NO: 2 - DNA, 31 bp, Artificial Sequence
  Synthetic
agatctgatggcgcgagggaaaaaaaaaaaa

SEQ ID NO: 3 - DNA, 10 bp, Artificial Sequence
  Synthetic
tcaggcagac

SEQ ID NO: 4 - DNA, 10 bp, Artificial Sequence
  Synthetic
agcatttcac

SEQ ID NO: 5 - DNA, 10 bp, Artificial Sequence
  Synthetic
cgacttagcc

SEQ ID NO: 6 - DNA, 10 bp, Artificial Sequence
  Synthetic
ggagttcaac

SEQ ID NO: 7 - DNA, 10 bp, Artificial Sequence
  Synthetic
ctacgagaac

SEQ ID NO: 8 - DNA, 10 bp, Artificial Sequence
  Synthetic
taggcgttac

SEQ ID NO: 9 - DNA, 10 bp, Artificial Sequence
  Synthetic
cgttctaatc

SEQ ID NO: 10 - DNA, 10 bp, Artificial Sequence
  Synthetic
gggaacgcgc

SEQ ID NO: 11 - DNA, 10 bp, Artificial Sequence
  Synthetic
ttgtagatcc

SEQ ID NO: 12 - DNA, 10 bp, Artificial Sequence
  Synthetic
tctatgggtc

SEQ ID NO: 13 - DNA, 19 bp, Artificial Sequence
  Synthetic
ggagcttgtgaattctggc

SEQ ID NO: 14 - DNA, 10 bp, Artificial Sequence
  Synthetic
tgcccaaggc

SEQ ID NO: 15 - DNA, 10 bp, Artificial Sequence
  Synthetic
cgtctcgatc

SEQ ID NO: 16 - DNA, 10 bp, Artificial Sequence
  Synthetic
tgcgccgagc

SEQ ID NO: 17 - DNA, 10 bp, Artificial Sequence
  Synthetic
atggatttac

SEQ ID NO: 18 - DNA, 10 bp, Artificial Sequence
  Synthetic
catgtttacc

SEQ ID NO: 19 - DNA, 10 bp, Artificial Sequence
  Synthetic
gtaacattac

SEQ ID NO: 20 - DNA, 10 bp, Artificial Sequence
  Synthetic
ggagttcaac

SEQ ID NO: 21 - DNA, 10 bp, Artificial Sequence
  Synthetic
ctttgtactc

SEQ ID NO: 22 - DNA, 10 bp, Artificial Sequence
  Synthetic
actaccgtgc

SEQ ID NO: 23 - DNA, 10 bp, Artificial Sequence
  Synthetic
atgaataagc

SEQ ID NO: 24 - DNA, 10 bp, Artificial Sequence
  Synthetic
aagaatttac

SEQ ID NO: 25 - DNA, 10 bp, Artificial Sequence
  Synthetic
caccattatc

SEQ ID NO: 26 - DNA, 10 bp, Artificial Sequence
  Synthetic
agagggaagc

SEQ ID NO: 27 - DNA, 10 bp, Artificial Sequence
  Synthetic
gtcggtggac

SEQ ID NO: 28 - DNA, 10 bp, Artificial Sequence
  Synthetic
gggatgatgc

SEQ ID NO: 29 - DNA, 10 bp, Artificial Sequence
  Synthetic
aaaacagggc

SEQ ID NO: 30 - DNA, 10 bp, Artificial Sequence
  Synthetic
attgatgatc

SEQ ID NO: 31 - DNA, 10 bp, Artificial Sequence
  Synthetic
gcaccctcac

SEQ ID NO: 32 - DNA, 10 bp, Artificial Sequence
  Synthetic
tggtaaaggc

SEQ ID NO: 33 - DNA, 10 bp, Artificial Sequence
  Synthetic
cacttagcgc

SEQ ID NO: 34 - DNA, 10 bp, Artificial Sequence
  Synthetic
aatgtagaac

SEQ ID NO: 35 - DNA, 10 bp, Artificial Sequence
  Synthetic
cgtgctccac

SEQ ID NO: 36 - DNA, 10 bp, Artificial Sequence
  Synthetic
acgcgcatac

SEQ ID NO: 37 - DNA, 10 bp, Artificial Sequence
  Synthetic
tggcgcactc

SEQ ID NO: 38 - DNA, 10 bp, Artificial Sequence
  Synthetic
ctacgagaac

SEQ ID NO: 39 - DNA, 10 bp, Artificial Sequence
  Synthetic
ctgtgacctc

SEQ ID NO: 40 - DNA, 10 bp, Artificial Sequence
  Synthetic
gaagaagacc

SEQ ID NO: 41 - DNA, 10 bp, Artificial Sequence
  Synthetic
taaatagttc

SEQ ID NO: 42 - DNA, 10 bp, Artificial Sequence
  Synthetic
tcctagcttc

SEQ ID NO: 43 - DNA, 10 bp, Artificial Sequence
  Synthetic
tccctaccac

SEQ ID NO: 44 - DNA, 10 bp, Artificial Sequence
  Synthetic
aggtcccgac

SEQ ID NO: 45 - DNA, 10 bp, Artificial Sequence
  Synthetic
taaggatgac

SEQ ID NO: 46 - DNA, 10 bp, Artificial Sequence
  Synthetic
ttgctcttac

SEQ ID NO: 47 - DNA, 10 bp, Artificial Sequence
  Synthetic
tccaacgacc

SEQ ID NO: 48 - DNA, 10 bp, Artificial Sequence
  Synthetic
tacatcttcc

SEQ ID NO: 49 - DNA, 10 bp, Artificial Sequence
  Synthetic
gttgcaggtc

SEQ ID NO: 50 - DNA, 10 bp, Artificial Sequence
  Synthetic
ccgggcttgc

SEQ ID NO: 51 - DNA, 10 bp, Artificial Sequence
  Synthetic
ggcgatagac

SEQ ID NO: 52 - DNA, 10 bp, Artificial Sequence
  Synthetic
cttctgaccc

SEQ ID NO: 53 - DNA, 10 bp, Artificial Sequence
  Synthetic
gtgcgacgcc

SEQ ID NO: 54 - DNA, 10 bp, Artificial Sequence
  Synthetic
agtaaacgac

SEQ ID NO: 55 - DNA, 10 bp, Artificial Sequence
  Synthetic
catcgcccgc

SEQ ID NO: 56 - DNA, 10 bp, Artificial Sequence
  Synthetic
aaaccgactc

SEQ ID NO: 57 - DNA, 10 bp, Artificial Sequence
  Synthetic
caaccatggc

SEQ ID NO: 58 - DNA, 10 bp, Artificial Sequence
  Synthetic
tctccattgc

SEQ ID NO: 59 - DNA, 10 bp, Artificial Sequence
  Synthetic
agcatttcac

SEQ ID NO: 60 - DNA, 10 bp, Artificial Sequence
  Synthetic
tacgcaaacc

SEQ ID NO: 61 - DNA, 10 bp, Artificial Sequence
  Synthetic
ataacctggc

SEQ ID NO: 62 - DNA, 10 bp, Artificial Sequence
  Synthetic
cgaagcgttc

SEQ ID NO: 63 - DNA, 10 bp, Artificial Sequence
  Synthetic
taggcgttac

SEQ ID NO: 64 - DNA, 10 bp, Artificial Sequence
  Synthetic
tgccaacatc

SEQ ID NO: 65 - DNA, 10 bp, Artificial Sequence
  Synthetic
cgacttagcc

SEQ ID NO: 66 - DNA, 10 bp, Artificial Sequence
  Synthetic
gtatgaaaac

SEQ ID NO: 67 - DNA, 10 bp, Artificial Sequence
  Synthetic
ttggcagggc

SEQ ID NO: 68 - DNA, 10 bp, Artificial Sequence
  Synthetic
tagatattgc